(19) United States
(12) Patent Application Publication
(10) Pub. No.: US 2013/0006264 A1
(43) Pub. Date: Jan. 3, 2013

(54) METHOD AND DEVICE FOR FACILITATING DELIVERY IN EVENT OF SHOULDER DYSTOCIA

(76) Inventor: David B. Klebanoff, Elkins Park, PA (US)

(21) Appl. No.: 13/172,112

(22) Filed: Jun. 29, 2011

(51) Int. Cl. A61B 17/42

(52) U.S. Cl. 606/124

(57) ABSTRACT

The present invention consists of devices and methods that may be used to facilitate childbirth in the event of shoulder dystocia. More specifically, the invention makes use of leverage to push the obstructed fetal shoulder below the pubic symphysis of the mother. Once shoulder dystocia is diagnosed, a person, usually a medical professional, would insert one of the described devices into the mother and against the obstructed shoulder of the child. The device would only be inserted as far as the guard member will allow. Using the described method, the medical professional would apply leverage to the device so as to separate the obstructed shoulder and the pubic symphysis, thus allowing the fetus to pass through the birth canal.

### BACKGROUND OF THE INVENTION

Shoulder dystocia is an extremely serious pregnancy complication where the fetus's anterior shoulder cannot pass below pubic symphysis of the mother. Shoulder dystocia is often recognized when the fetal head is delivered, but soon retracts back inside the mother. If not remedied quickly, shoulder dystocia may result in the death of the fetus due to asphyxiation.

Several inventions and maneuvers exist for remedying shoulder dystocia. The most common method of remedy is the McRoberts maneuver. This maneuver requires the mother to pull her legs tightly into her abdomen. US Patent Publ. 2007/0272255 describes an invention that facilitates this maneuver. This invention consists of straps that are attached to the mothers wrists and feet. The straps allow the mother to invoke the McRoberts maneuver without the aid of nurses.

Other maneuvers include: the Woods' screw maneuver, which involves the rotation of the anterior shoulder; suprapubic pressure, or pressure on the lower abdomen; posterior pressure on the obstructed shoulder; Jacquemier's maneuver, where the posterior shoulder is delivered allowing the attached arm to be gently pulled; and the Gaskin maneuver, where the mother positions herself on her hands and knees and arches her back to widen the pelvis and facilitate passage of the anterior shoulder.

Some inventions in the art allow for the facilitation of delivery via a vacuum suction device which can pull on the obstructed shoulder. US Patent Publ. 2007/0162049 and 2009/0270879 describe suction cups designed to fit the shape of the shoulder, thus allowing the medical professional to pull on the shoulder instead of or in addition to the fetal head. Only pulling on the fetal head can result in injury to the brachial plexus of the fetus. Brachial plexus may also result from use of the maneuvers listed above. US Patent Publ. 2006/0074364 describes a device that reduces the risk of brachial plexus via a neck brace that limits the lateral movement of the fetal head.

While the conventional maneuvers and prior art devices do facilitate successful delivery in many cases, none result in successful delivery in all occurrences.

It is therefore an object of the invention to provide a device and method which improves the rate of successful delivery in the event of shoulder dystocia.

### SUMMARY OF THE INVENTION

These, and other objects as will become apparent from the following disclosure and drawings, are achieved by the present invention which comprises: 1) an obstetric prying device designed to facilitate delivery in the event of shoulder dystocia by allowing for the application of leverage against the anterior shoulder of the fetus; and 2) a method of using leverage to facilitate delivery in such event. The first aspect of the invention is a device that consists of a lever member having a guard member positioned on said top side of said lever member wherein said guard member is of sufficient height to limit the depth of vaginal insertion.

In another aspect, the invention comprises method of facilitating childbirth in an event of shoulder dystocia, wherein the anterior shoulder of a fetus cannot pass below the pubic bone of a mother, comprising the steps of inserting a lever having a distal end and a proximal end into said mother's vagina; positioning said distal end between said anterior shoulder and said pubic bone; and exerting leverage on said proximal end, using said pubic bone as a fulcrum, so as to cause said distal end to push against said anterior shoulder with sufficient pressure to allow said anterior shoulder to pass below said pubic bone.

In some embodiments of the device the lever member is between 12 and 20 inches in length and between 3 and 5 inches in width at its widest point; the guard member is between 2 and 4 inches high; the distal end of the lever member extends 4 to 6 inches beyond the guard member; the lever member is between ⅛ and ⅜ inches thick; the edges of the lever member are rounded; and the device includes both a top handle and a proximal handle with the proximal handle oriented at an angle between 10 and 30 degrees below the plane of the lever member. In preferred embodiments of the device, the lever member has a shoehorn-like shape, gently curved so as to allow the concave side to fit around the fetal shoulder. In some embodiments the lever member comprises a tubular proximal structure and a concave, shoehorn-like shaped distal structure with the guard member positioned on the distal structure.

The preferred embodiment of the method is use of the previously mentioned device where the device is inserted into the vagina along the fetus's back—for example, at the 5 o'clock or 7 o'clock position—and then maneuvered to the area between the pubic bone and the fetus's anterior shoulder before leverage is applied. During insertion of the device at the 5 o'clock or 7 o'clock position, the doctor may gently push the fetal head to the side in order to facilitate insertion and reduce the risk of harm to the fetus. While leverage is applied so as to push the obstructed fetal shoulder below the mother's pubic bone, the same or another doctor or nurse may gently pull on the fetal head to further facilitate delivery.

### BRIEF DESCRIPTION OF THE DRAWINGS

FIG. 1 is a perspective view of an embodiment of a device according to the invention.

FIG. 2 is a cross-sectional side view through II-II in FIG. 1.

FIG. 3 is a side elevational view of the embodiment in FIG. 1.

FIG. 4 is a top plan view of the embodiment of FIG. 1.

FIG. 5 is a front perspective view of a surgeon moving a fetus's head to one side during a delivery to allow insertion of the embodiment of FIG. 1 at 7 o'clock.

FIG. 6 is a front perspective view of insertion of the embodiment of FIG. 1 at 7 o'clock, in phantom, and rotating said embodiment to 12 o'clock, in solid lines.

FIG. 7 is a side view, partially in cross-section, of a device according to the invention, being used to press down on the anterior shoulder of a fetus during a delivery.

FIG. 8 is a front elevational view of the embodiment in FIG. 1 with the proximal handle not shown.

FIG. 9 is a front elevational view of a second embodiment of the invention wherein the lever member comprises a tubular proximal structure and a concave distal structure with the guard member positioned on the distal structure.

### DETAILED DESCRIPTION

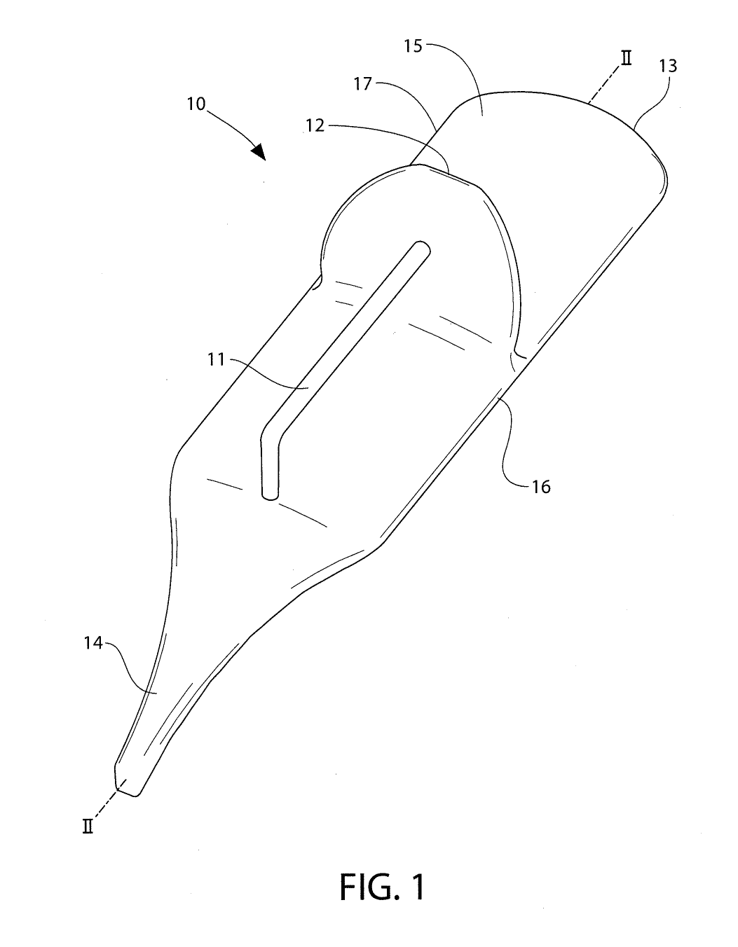

Referring now in detail to the drawings, FIG. 1 is a perspective view of the top side 15 of an obstetric prying device 10 according to the invention. A guard member 12 and a top handle 11 protrude from the top side 15 of the lever member 17. A proximal handle 14 extends from the proximal end of the device 10. In the illustrated embodiment, the device 10 has rounded edges 16. In some embodiments the device has a length between 12 and 20 inches from the distal end 13 to the end of the proximal handle 14; and a width between 3 and 5 inches at its widest point. In some embodiments the top handle 11 can be deleted.

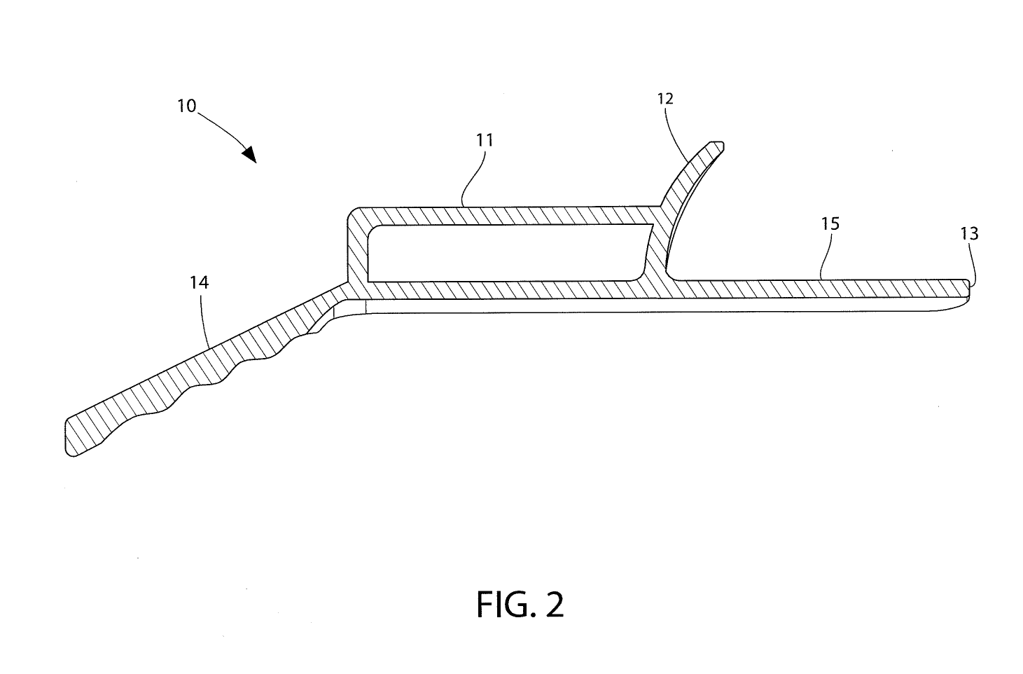

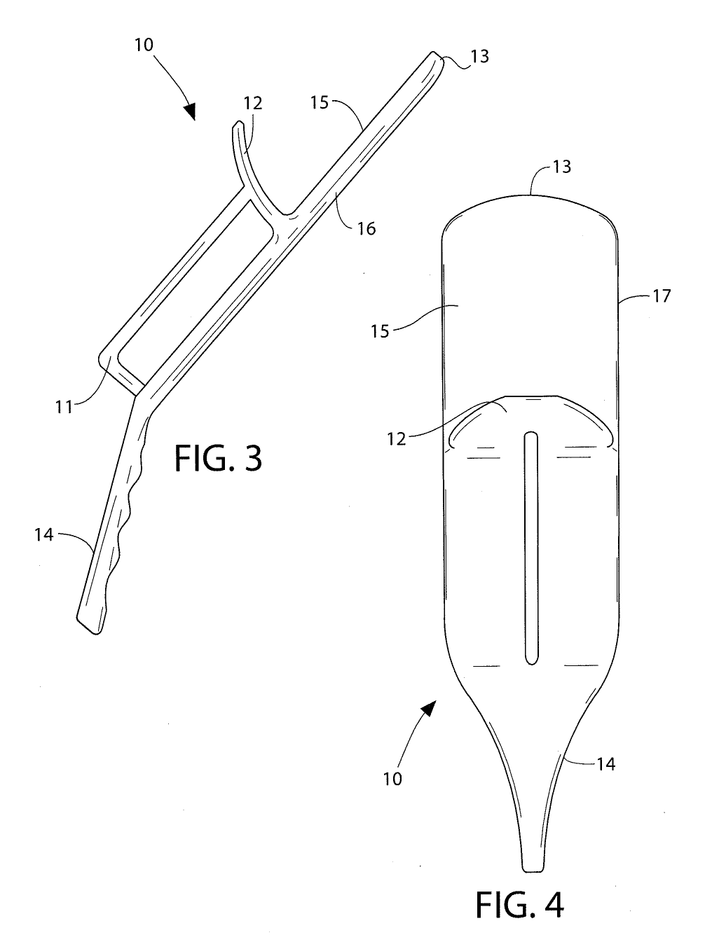

FIG. 2 is a cross-sectional side view through II-II of FIG. 1 and FIG. 3 is a side elevational view of the obstetric prying device 10 of FIG. 1. In some embodiments, the guard member 12 is between 2 and 4 inches high; the distance between the distal end 13 and the guard member 12 is between 4 and 6 inches; the proximal handle 14 is rounded and adapted to be gripped by a hand; and said proximal handle 14 is oriented at an angle between 10 and 30 degrees below the plane of the top side 15.

FIG. 4 is a top view of the device 10 of FIG. 1.

In the preferred embodiment the entire device 10 is made from surgical stainless steel, and the lever member 17 has a shoehorn-like shape wherein a side-to-side cross-section would be concave from a bottom view or convex from a top view.

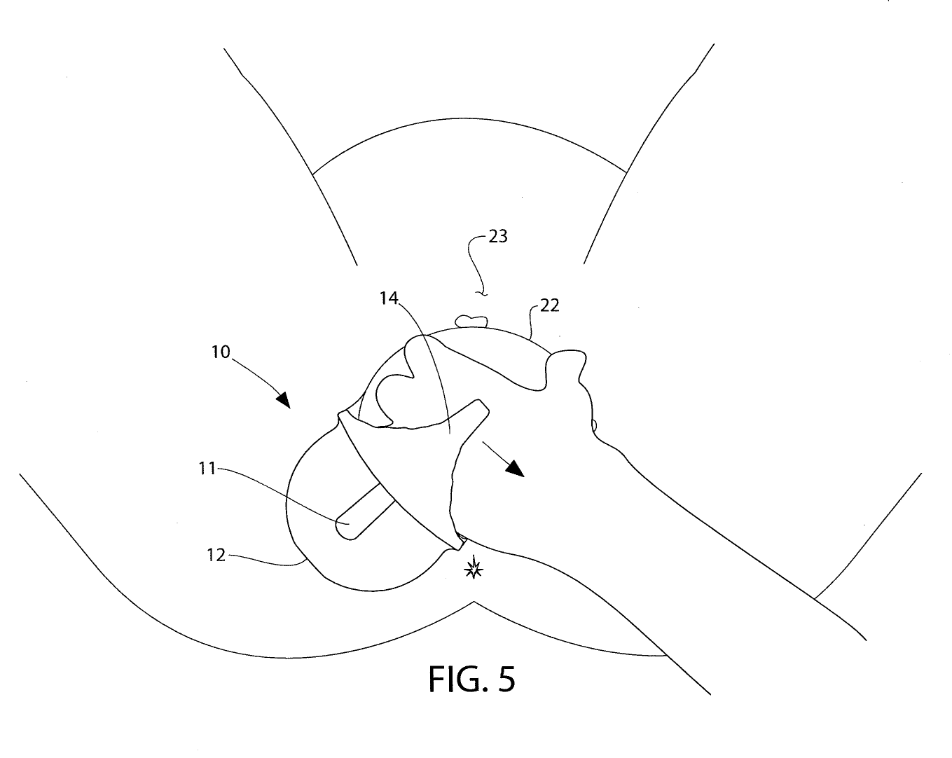

In FIG. 5, the doctor gently moves the fetal head 22 very gently and slightly to the side to facilitate insertion of the device 10 of FIG. 1 into the mother's vagina and reduce the risk of injury to the fetus. The device 10 is illustrated as being inserted at 7 o'clock in FIG. 5. The guard member 12 prevents the device 10 from slipping too far inside the mother.

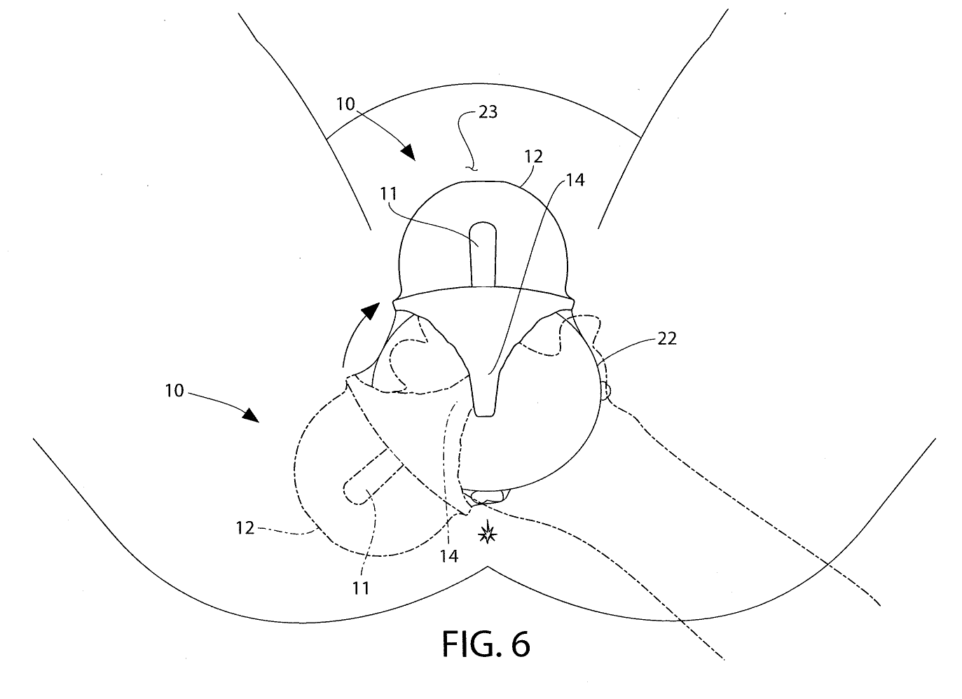

FIG. 6 depicts the rotation of the device 10 of FIG. 1 from 7 o'clock to 12 o'clock. The device 10 in phantom illustrates how the device 10 was initially inserted as shown in FIG. 5. The device 10 in solid lines shows how the device 10 is oriented after rotation, so that the device 10 is above the fetal head and the guard member 12, abuts the pubic bone 23.

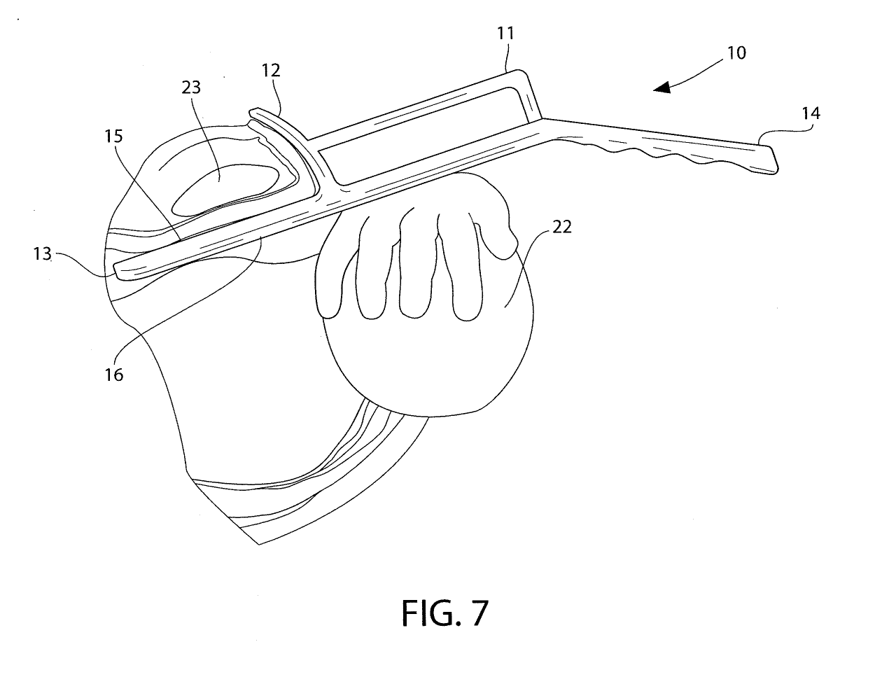

In FIG. 7, the device 10 of FIG. 1 is fully inserted into the mother's vagina so that the guard member 12 abuts the pubic bone 23. By gently pulling upward on the proximal handle 14, top handle 11, or both, the doctor uses the pubic bone 23 as a fulcrum so that the distal end 13 of the device 10 pushes down on the fetal shoulder and thereby permit the anterior shoulder to pass without obstruction. The rounded edges 16 of the device 10 reduce the risk of injury to the mother and fetus.

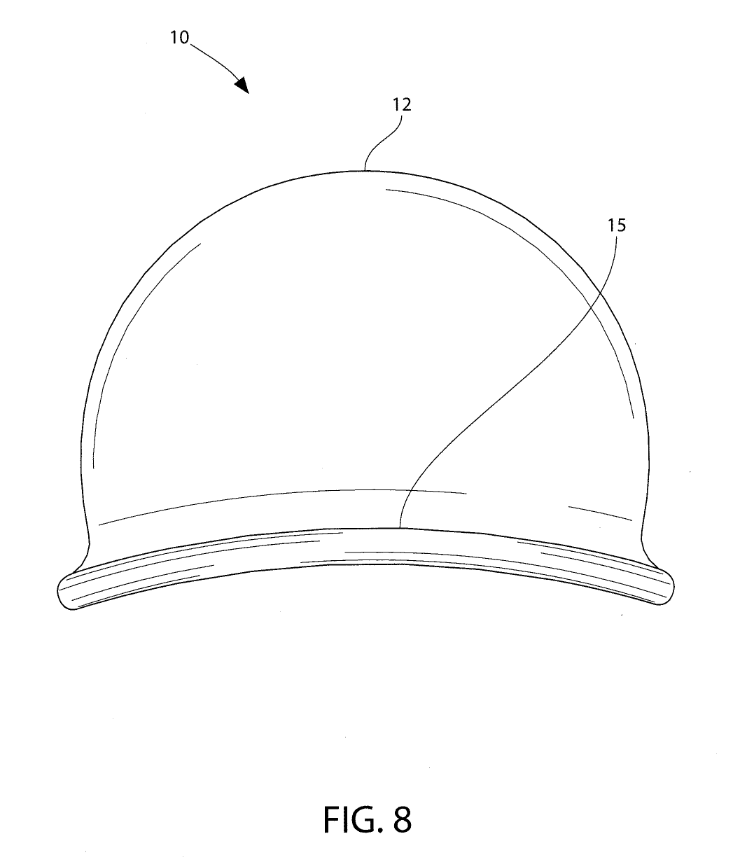

FIG. 8 is a front elevational view of the device 10 from FIG. 1 with the proximal handle not shown. This view depicts the device 10 from the distal end 13 and shows concave curvature of the bottom surface. Guard member 12 is seen in this view.

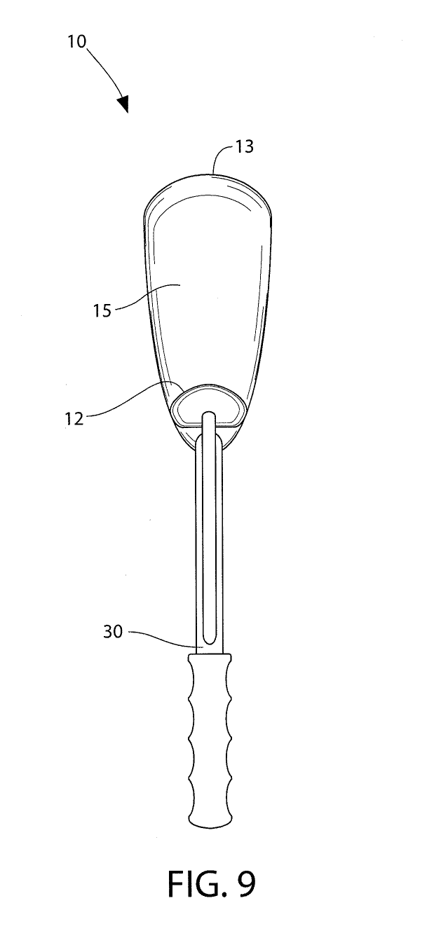

FIG. 9 a front elevational view of a second embodiment of the invention wherein the lever member 10 comprises a tubular proximal structure 30 and a concave distal structure 15 with the guard member 12 positioned on the distal structure.

The present invention, therefore, is well adapted to carry out the objects and attain the ends and advantages mentioned, as well as others inherent therein. While the invention has been depicted and described and is defined by reference to particular preferred embodiments of the invention, such references do not imply a limitation on the invention, and no such limitation is to be inferred. The invention is capable of considerable modification, alteration and equivalents in form and function, as will occur to those ordinarily skilled in the pertinent arts. The depicted and described preferred embodiments of the invention are exemplary only and are not exhaustive of the scope of the invention. Consequently, the invention is intended to be limited only by the spirit and scope of the appended claims, giving full cognizance to equivalents in all respects.

What is claimed is:

1. An obstetric prying device comprising:
a lever member having a distal end, a proximal end, and a top side; and
a guard member positioned on said top side of said lever member wherein said guard member is adapted to limit the depth of vaginal insertion of the device.

2. The obstetric prying device of claim 1, further including a top handle on said top side of said lever member.

3. The obstetric prying device of claim 1, further including a proximal handle on said proximal end of said lever member.

4. The obstetric prying device of claim 1, wherein said device is made from surgical stainless steel, plastic, or a combination of surgical stainless steel and plastic.

5. The obstetric prying device of claim 1, wherein the height of said guard member is between 2 and 4 inches.

6. The obstetric prying device of claim 1, wherein the width of said lever member at its widest point is between 3 and 5 inches.

7. The obstetric prying device of claim 1, wherein the length of said lever member is between 12 and 20 inches.

8. The obstetric prying device of claim 1, wherein the distance between said guard member and said distal end of said lever member is between 4 and 6 inches.

9. The obstetric prying device of claim 1, wherein the thickness of said lever member is between ⅛ and ⅜ inches.

10. The obstetric prying device of claim 1, wherein the edges of said lever member are rounded.

11. The obstetric prying device of claim 1, further including a proximal handle on said proximal end of said lever member, wherein said proximal handle is oriented at an angle between 10 and 50 degrees downward in relation to said top side of said lever member.

12. The obstetric prying device of claim 1, further including a proximal handle on said proximal end of said lever member, wherein said proximal handle has an opening to receive fingers of a hand.

13. The obstetric prying device of claim 1, further including a proximal handle on said proximal end of said lever member, wherein said proximal handle has a rounded shape adapted to be gripped by a hand.

14. The obstetric prying device of claim 1, further including a top handle on said top side of said lever member, wherein said top handle has an opening to receive fingers of a hand.

15. The obstetric prying device of claim 1, further including a top handle on said top side of said lever member, wherein said top handle has a rounded shape adapted to be gripped by a hand.

16. The obstetric prying device of claim 1 wherein the lever member comprises a tubular proximal structure and a concave distal structure with the guard member positioned on the distal structure.

17. The obstetric prying device of claim 1, wherein:
said device further includes a proximal handle on said proximal end of said lever member, wherein said proximal handle has a rounded shape adapted to be gripped by a hand, and is oriented at an angle between 10 and 50 degrees downward in relation to said top side of said lever member;
said device further includes a top handle on said top side of said lever member, wherein said top handle has an opening to receive fingers of a hand;
the height of said guard member is between 2 and 4 inches;
the width of said lever member at its widest point is between 3 and 5 inches;
the length of said lever member is between 12 and 20 inches;
the distance between said guard member and said distal end of said lever member is between 4 and 6 inches;
the thickness of said lever member is between ⅛ and ⅜ inches;
the edges of said lever member are rounded; and
said device is made from surgical stainless steel.

18. A method of facilitating childbirth in an event of shoulder dystocia, wherein the anterior shoulder of a fetus cannot pass below the pubic bone of a mother, comprising the steps of:
inserting a lever having a distal end and a proximal end into said mother's vagina;
positioning said distal end between said anterior shoulder and said pubic bone; and
exerting leverage on said proximal end, using said pubic bone as a fulcrum, so as to cause said distal end to push against said anterior shoulder with sufficient pressure to allow said anterior shoulder to pass below said pubic bone.

19. The method of claim 17, wherein:
said lever is comprised of a lever member having a distal end, a proximal end, a top side, and a guard member positioned on said top side of said lever member, wherein said guard member is of sufficient height to limit the depth of vaginal insertion; and
said guard member limits the distance at which the lever can be inserted into the vagina and abuts said pubic bone of said mother when leverage is applied to said proximal end.

20. The method of claim 17, wherein:
said distal end of said lever is inserted into said vagina along said fetus's back, and then maneuvered between said anterior shoulder and said pubic bone prior to exerting leverage.

21. The method of claim 17, wherein:
said lever is comprised of a shoehorn-shaped lever member having a distal end, a proximal end, a top side, and a guard member positioned on said top side of said lever member, wherein said guard member is of sufficient height to limit the depth of vaginal insertion;
said guard member limits the distance at which the lever can be inserted into the vagina and abuts said pubic bone of said mother when leverage is applied to said proximal end; and
said distal end of said lever is inserted into said vagina along said fetus's back, and then maneuvered between said anterior shoulder and said pubic bone prior to exerting leverage.
